(19) United States
(12) Patent Application Publication
(10) Pub. No.: US 2014/0378988 A1
(43) Pub. Date: Dec. 25, 2014

(54) SUPPORTED RETRIEVAL DEVICE AND RELATED METHODS OF USE

(71) Applicant: Boston Scientific Scimed, Inc., Maple Grove, MN (US)

(72) Inventors: Samuel RAYBIN, Marlborough, MA (US); Paul SMITH, Smithfield, RI (US); Naroun SUON, Lawrence, MA (US); Jeffrey BEAN, Fitchburg, MA (US); Daniel LANG, North Attleboro, MA (US); John GOLDEN, Norton, MA (US)

(73) Assignee: Boston Scientific Scimed, Inc., Maple Grove, MN (US)

(21) Appl. No.: 14/302,166

(22) Filed: Jun. 11, 2014

Related U.S. Application Data
(60) Provisional application No. 61/837,360, filed on Jun. 20, 2013.

(51) Int. Cl. A61B 17/221

(52) U.S. Cl. CPC A61B 17/221; A61B 2017/2212
USPC 606/113

(57) ABSTRACT

A medical device for manipulating a tissue is disclosed. The device includes an elongate shaft having a proximal end, a distal end, and a first lumen between the proximal end and the distal end, which has an opening in communication with the first lumen. The elongate shaft further includes a second lumen or aperture disposed in an external surface of the elongate shaft. The device further includes: a support truss, a snare loop, and an actuation member having a distal end. The actuation member is movably disposed within the first lumen. The support truss is configured to be received within the first lumen, second lumen, or aperture such that a proximal end of the support truss is operably connected to the elongate shaft adjacent a proximal portion of the first lumen, second lumen, or aperture. The snare loop has a first portion connected to a distal end of the support truss and a second portion connected to the distal end of the actuation member.

### CROSS-REFERENCE TO RELATED APPLICATIONS

This application claims the benefit of priority from U.S. Provisional Application No. 61/837,360, filed on Jun. 20, 2013, the entirety of which is incorporated by reference herein.

### FIELD

Embodiments of the present disclosure relate generally to medical devices and procedures. In particular, embodiments of the present disclosure relate to medical devices to perform tissue resection and/or retrieval during minimally invasive medical procedures.

### BACKGROUND

Tissue resection procedures, such as colonoscopy and polypectomy, are carried out by inserting introduction sheaths, such as endoscopes or laparoscopes, into the body of a patient through incisions or natural anatomical openings (e.g., oral, vaginal, and/or anal cavities). Commonly, such devices employ snares, typically designed as loops, for performing tissue resection procedures. Snares have a tendency to slip off targeted tissue, however, often requiring repeated efforts to capture the tissue before the resection procedures can be successfully performed.

One common approach is to apply a downward force on the snare in an effort to improve traction between the snare and the unwanted tissue. This downward force is usually limited due to a lack of stiffness in the snare loop, however, causing the distal end of the snare loop to deflect away from the tissue. In order to control this deflection, the downward force may continue to be applied or it may be increased until the tissue is snared. Continued or increased applied force increases the possibility of accidentally damaging surrounding tissues, as well as increasing the time required to complete a procedure. In addition, it may be sometimes difficult to apply the necessary downward forces due to geometric and physical limitations.

In another approach, the rigidity of the snare loop is increased to reduce the amount of deflection caused by the applied force. However, such an increase in rigidity may reduce the required flexibility of the snare loop and interfere with its functioning.

Therefore, there exists a need for an improved snare with better traction and control capabilities, thereby allowing consistent capture, excision, and/or removal of unwanted tissue.

### SUMMARY

Aspects of the present disclosure relate to medical devices and methods for performing tissue-ensnaring procedures during, e.g., tissue resection from inside a patient's body.

In accordance with an aspect of the present disclosure, the medical device may include an actuation member, a support truss, a snare loop, and an elongate shaft having a proximal end, a distal end, and a lumen between the proximal and the distal ends. The elongate shaft includes a slot disposed in its external wall, configured to receive a support truss. The proximal end of the support truss may be operably connected to the shaft adjacent a proximal portion of the slot, which has a distal opening in communication with the lumen. The lumen includes a movable actuation member, whose distal end may be connected to a portion of a snare loop and another portion of the snare loop may be secured to the distal end of the support truss. Further, the snare loop may be configured to transition between a collapsed configuration and an expanded configuration. The medical device may be introduced within the body and advanced to a desired location, where the snare loop may be extended to receive the tissue for manipulation.

In some aspects, the support truss may be collapsible to fit within the lumen of the elongate shaft, eliminating the need for the slot. In such examples, in the collapsed configuration, the support truss, snare loop, and actuation member may fit within the lumen of the elongate shaft. In the expanded configuration, the support truss, snare loop, and actuation member may extend from a single opening of the elongate shaft, such as the distal opening. Specifically, the elongate shaft may be fully annular or cylindrical (e.g., without a through-slot), along its length up to a distal opening at a distal end of the elongate shaft. In this example, the support truss, snare loop, and actuation member may be collapsed into a delivery configuration having an outer diameter that is smaller than an inner diameter of the lumen of the elongate shaft. When some or all of the collapsed support truss, snare loop, and actuation member are extended distally through the distal opening, they may expand into a deployed configuration having a cross section that is, in one or more places, wider than the inner diameter of the lumen of the elongate shaft.

In some examples, the device may include one or more of the following additional features: the support truss may be configured to advance distally within the slot and/or may be pivotally attached at the proximal end of the slot; the truss may be substantially rigid; the actuation member may be a single or braided wire, which may be configured to conduct energy and cauterize tissue. The device may further include an instrument configured to be disposed within one of a plurality of working channels of an elongate introduction sheath, which may include imaging and illumination devices. Still further, a distal portion of the support truss may include a first extension arm attached to the support truss forming an angle relative to a second extension arm attached to the support truss; the first extension arm may be configured to be pivotable relative to the second extension arm; and each of the first and the second extension arms may attach to at least one rod configured to extend distally from a distal end of the extension arm.

In another aspect, a medical device for manipulating a tissue may include: an elongate member defining an elongate opening at a distal end leading to a distally facing opening; a substantially rigid member disposed within the elongate opening; and an expandable wire loop configured to transition between a collapsed configuration and an expanded configuration, wherein a portion of the expandable wire loop is connected to the substantially rigid member.

In accordance with another aspect, a method for manipulating tissue may include: introducing a medical device into the body, the medical device comprising an elongate shaft having a proximal end, a distal end, and a first lumen extending therebetween, wherein the distal end of the elongate shaft includes an opening in communication with the lumen, and wherein the elongate shaft further includes a second lumen or aperture disposed in an external surface of the elongate shaft; advancing an actuation member of the device to advance the medical device to a desired location within the body, the actuation member movably disposed within the first lumen, wherein the actuation member includes a distal end; advancing a snare loop of the device to receive tissue, the snare loop having a first portion connected to a distal end of a support truss and a second portion connected to the distal end of the actuation member; and supporting the snare loop with the truss support while advancing the snare loop, the truss support configured to be received within the first lumen, the second lumen, or the aperture, wherein a proximal end of the support truss is operably connected to the shaft adjacent a proximal portion of the first lumen, second lumen, or aperture.

Additional objects and advantages of the present disclosure will be set forth in part in the description which follows, and in part will be understood from the description, or may be learned by practice of the claimed invention. The objects and advantages of the claimed invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 is a perspective view of a distal portion of an exemplary retrieval device in a collapsed configuration, according to an embodiment of the present disclosure.

FIG. 2A is a perspective view of the exemplary retrieval device of FIG. 1 in an open configuration.

FIG. 2B is a side view of the exemplary retrieval device of FIG. 1 in the expanded configuration.

FIG. 2C is a distal end view of the exemplary retrieval device for use of FIG. 1 in the expanded configuration.

FIGS. 3A and 3B illustrate actuation of the exemplary retrieval device from the collapsed configuration of FIG. 1 to the expanded configuration of FIGS. 2A-2C according to a first embodiment of the present disclosure.

FIGS. 4A, 4B, and 4C illustrate operation of an exemplary retrieval device according to a second embodiment of the present disclosure.

FIGS. 5A, 5B, 5C, and 5D illustrate operation of an exemplary retrieval device according to a third embodiment of the present disclosure.

### DESCRIPTION OF THE EMBODIMENTS

Reference will now be made in detail to embodiments of the present disclosure, examples of which are illustrated in the accompanying drawings. Wherever possible, the same reference numbers will be used throughout the drawings to refer to the same or like parts. The term “distal” refers to the end farthest away from a user when introducing a device into a patient. By contrast, the term “proximal” refers to the end closest to the user when placing the device into the patient.

### Overview

Embodiments of the present disclosure relate to medical devices used to retrieve and/or sever unwanted tissue as well as other unwanted material, such as stones, within a patient's body. For example, embodiments of the disclosed device may facilitate removal of unwanted tissue, such as, cancerous polyps or lesions, from within a patient's body, including tissue disposed on, e.g., the mucosal walls of the colon, esophagus, stomach, or duodenum. A physician may also desire to resect tissue in order to conduct a biopsy or other examination. It should be noted that devices presented in the present disclosure can be used both for retrieving and for severing target tissue or objects. For convenience, the medical devices discussed here will be referred to as “retrieval devices,” it being understood that such devices are equally useful for severing unwanted tissue.

In some embodiments, a retrieval device may include an elongate shaft having a distal opening, a support truss, and a snare loop. The elongate shaft may include a slot located in an exterior wall of the shaft and proximal to the distal opening. The support truss may be configured to be received in the slot such that a proximal end of the support truss is attached (e.g., pivotally) to a proximal end of the slot. The snare loop may have a first portion attached to a distal end of the support truss and a second portion associated with the elongate shaft. In some embodiments, the second portion may alternatively be operably coupled to an actuation element. The support truss may provide stiffness to the snare loop and improve traction between the snare loop and material (e.g., tissue) targeted for retrieval and/or removal.

### Exemplary Embodiments

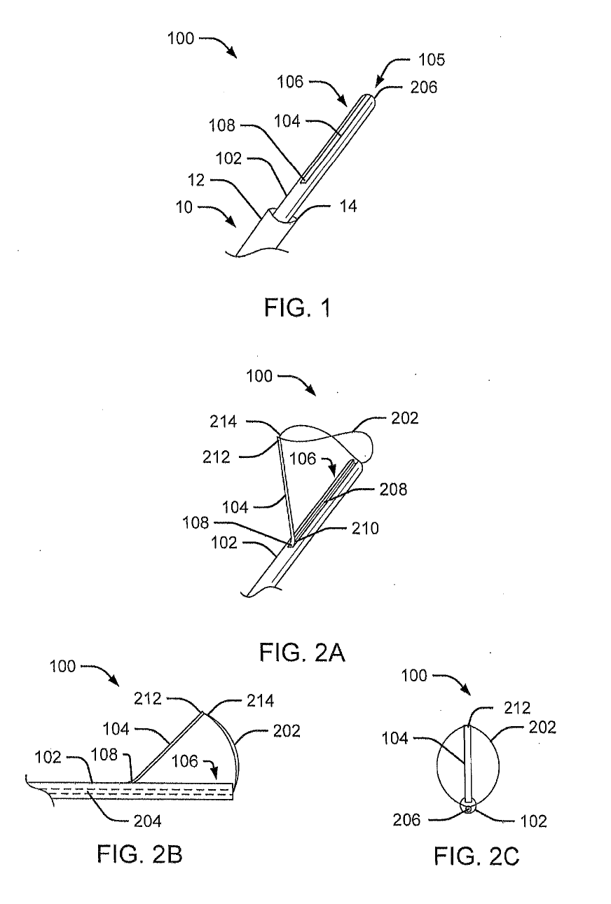

FIG. 1 is a perspective view of a distal portion of an exemplary retrieval device 100 in a collapsed configuration, according to an embodiment of the present disclosure. The retrieval device 100 may be configured to be introduced into a patient's body through an incision or a suitable natural opening. In addition, the retrieval device 100 may be configured to be advanced to a desired location within a patient's body with the aid of a suitable introduction sheath, such as, an endoscope 10. Endoscope 10 may include a steerable elongate sheath 12 having a distal end 14, a proximal end (not shown) and one or more working channels (not referenced) extending therebetween.

As discussed above, endoscope 10 may include one or more working channels, through which an operator may introduce one or more medical devices to extend out of the distal end 14 of elongate sheath 12. For example, during a resectomy procedure, an operator may introduce a suction device into one channel and another instrument, such as, retrieval device 100, into another channel. Additionally, during the procedure, the operator may insert a light source, a camera, an injector, and/or a morcellator within the one or more channels. The proximal end of the elongate sheath 12 may be connected to a hub assembly or handle (not shown) for operating endoscope 10. The handle may be ergonomically designed and may include a variety of components, such as steering controls, for selectively positioning distal end 14. Further, the handle may include one or more ports in communication with the one or more working channels for inserting one or more medical devices into endoscope 10.

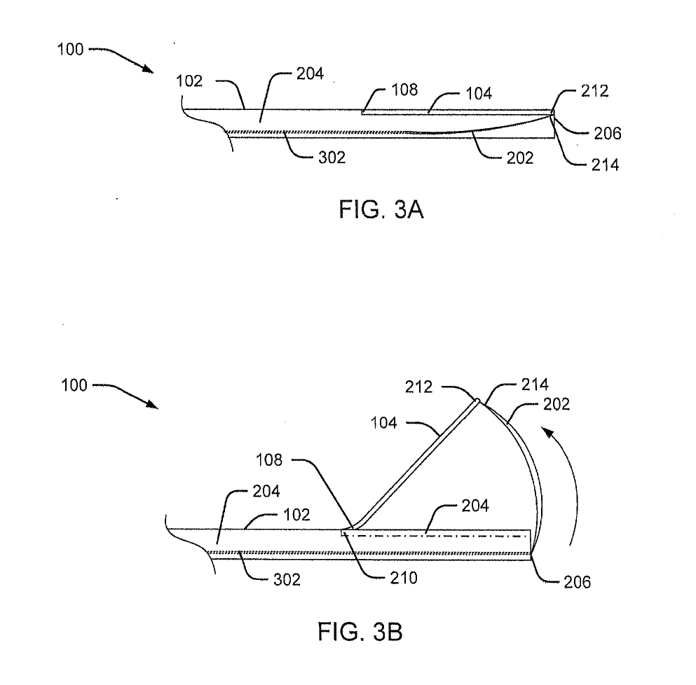

A retrieval device, such as the retrieval device 100, may be defined as a device used for severing and/or capturing unwanted tissue or other material inside the body of a patient. As shown in FIGS. 1 and 2A-2C, the retrieval device 100 may include an elongate shaft 102, a support truss 104, and a snare loop 202 (FIG. 2A-2C). The elongate shaft 102 includes a proximal end (not shown), distal end 105, and a lumen 204 (FIG. 2A-2C) extending between the proximal end and the distal end 105. The lumen 204 may be in communication with a distal opening 206 (FIG. 2A-2C) at a distal end of the shaft 102. The lumen 204 may extend the entire length of the shaft 102. However, the shaft 102 may include a number of lumens and/or corresponding openings for a variety of purposes, such as inserting medical devices, deploying an optical lens, or the like. The elongate shaft 102 may be configured to be steerable independently of endoscope 10 using, for example, a control wire.

The elongate shaft 102 may further include a slot 208 (FIG. 2A-2C) formed in an external wall 106 of the shaft 102. Slot 208 may extend entirely through external wall 106 of shaft 102 such that slot 208 is in communication with lumen 204. In some embodiments, however, slot 208 may only extend partially through external wall 106. Further, slot 208 may be disposed on a distal portion of shaft 102. As depicted, slot 208 may extend distally from a location proximal to the distal end of shaft 102. In such embodiments, the distal opening 206 may be in communication with the slot 208, which may be any suitable opening and/or recess configured to receive a portion of truss 104. In some embodiments, slot 208 may be configured to receive an entirety of a support truss 104 therein.

In some embodiments, the support truss 104 may be collapsible to fit within the lumen 204 of the elongate shaft 102, eliminating the need for the slot 208. In such embodiments, in the collapsed configuration, the support truss 104, snare loop 202, and actuation member may fit within the lumen 204 of the elongate shaft 102. In the expanded configuration, the support truss 104, snare loop 202, and actuation member may extend from a single opening of the elongate shaft 102, such as the distal opening 206. Specifically, the elongate shaft 102 may be fully annular or cylindrical (e.g., without a through-slot), along its length up to a distal opening at a distal end of the elongate shaft 102. In this embodiment, the support truss 104, snare loop, 202 and actuation member may be collapsed into a delivery configuration having an outer diameter that is smaller than an inner diameter of the lumen 204 of the elongate shaft 102. When some or all of the collapsed support truss 104, snare loop 202, and actuation member are extended distally through the distal opening, they may expand into a deployed configuration having a cross section that is, in one or more places, wider than the inner diameter of the lumen 204 of the elongate shaft 102.

Further, the support truss 104 may be, for example, a rigid member configured to support the snare loop 202 to reduce unwanted deflection of the loop 202 during use. The support truss 104 may include a proximal portion, such as, the proximal end 108, which may be detachably connected, permanently coupled, or formed integral with a proximal portion, such as, the proximal end 210 (FIGS. 2A-2B) of the slot 208 using any of the variety of mechanisms known in the art. Examples of these mechanisms include, but are not limited to, welding, molding, a snap fit, a screw fit, and gluing, any of which may be chosen depending on the material from which the support truss 104 is made.

In one example, the proximal end 108 of support truss 104 may be pivotally attached to the proximal end 210 of the slot 208 using one or more swivels or hinges. In another example, the truss 104 may be formed integrally with elongate shaft 102, joined by a flexible strip of material enabling it to pivot about its proximal end 108. The degree of flexibility of the support truss 104 may be predetermined based on a variety of factors. Such factors include, but are not limited to, (1) the ability of the truss 104 to retract into the slot 208 within the wall 106 of the shaft 102 after the truss 104 has been pivoted away from the shaft 102, and (2) the amount of stiffness to be imparted to the snare loop 202.

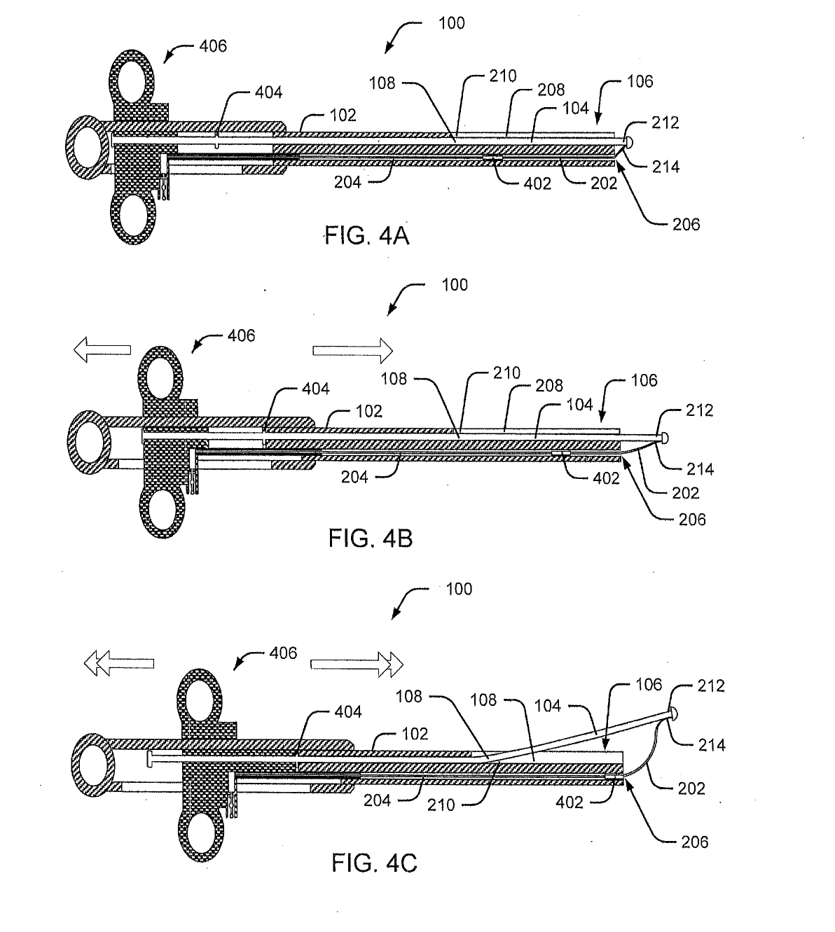

In one embodiment, the snare loop 202 may be a wire configured to sever or retrieve unwanted tissue. The snare loop 202 wire may include a braided wire, multiple wires, or other suitable wires known to those skilled in the art. In addition, the material employed to manufacture such wires may include, but not be limited to, a rigid, a flexible, or a semi-rigid material. Exemplary materials may include metals, polymers, composites, alloys, or the like. Further, the snare loop 202 may be configured in a variety of shapes, such as a continuous loop, multiple loops, a basket, or the like. In one implementation, the snare loop 202 may be substantially circular in shape. Other embodiments may present ellipsoid or other shapes and sizes. As in known types of snare loops, snare loop 202 may be connected to truss 104 and to a snare actuating element 302 (FIG. 3A-3B), discussed later. The snare loop 202 may be attached to the support truss 104 at any location, such as distally, along the circumference of the snare loop 202.

In a collapsed configuration, as shown in FIG. 1, the snare loop 202 may be located inside the lumen 204 of the shaft 102, while the support truss 104 may be located within a slot 208 of the shaft 102 such that the diameter of the retrieval device 100 is approximately the same as that of the shaft 102. Further, in the collapsed configuration, a cross-section of the shaft 102, along with the truss 104 located within the slot 208, may be configured to be substantially circular; however, other suitable cross-sectional shapes, for example, elliptical, oval, polygon, irregular, etc., may be employed.

FIG. 2A is a perspective view, FIG. 2B is a side view, and FIG. 2C is a distal end view of the exemplary retrieval device 100 in an expanded configuration according to an embodiment of the present disclosure. The retrieval device 100 includes the elongate shaft 102 having a lumen 204 therein for carrying the snare loop 202 and the slot 208 sized for receiving the support truss 104.

To reach an expanded configuration, truss 104 may pivot radially away from slot 208 and away from elongate shaft 102, drawing snare loop 202 from the lumen 204. It is contemplated that truss 104 may be biased relative to the shaft 102 such that upon deployment of snare loop 202 out of shaft 102, the truss 104 may pivot and assist in drawing snare loop 202 from the lumen 204. In addition or alternatively, snare loop 202 may cause truss 104 to pivot as snare loop 202 is deployed out of the lumen 204 by, for example, the actuation element 302 as described in more detail below. The wire from which snare loop 202 is formed may be sufficiently stiff to preferentially assume a set shape when open, such as a circle or ellipse. Accordingly, resultant stresses in unanchored portions of the snare loop 202, such as those which are not connected to a snare actuating element 302 (FIG. 3A-3B) or support truss 104. Such resultant stresses may cause the snare loop 202 to bow outwards away from the junction of the shaft 102 and the support truss 104. Consequently, the snare loop 202 may bow outside of a single plane, though alternatively, the snare loop 202 may remain in a single plane. Further, since the distal end 214 of the snare loop 202 is attached to the distal end 212 of the truss 104, the snare loop 202 may be configured to pivot about its proximal end in a direction relative to the angular position of the support truss 104 with respect to the shaft 102.

When the snare loop 202 pivots about its proximal end under the influence of the support truss 104, the point of connection between the snare loop 202 and the support truss 104 at their distal ends 214, 212 may include a pivoting joint for reducing strain on the snare loop 202 and the truss 104 near the point of connection. Those skilled in the art may appreciate that the support truss 104 may be made to pivot in any plane other than the longitudinal plane of the shaft 102. Further, the attachment of the truss 104 to the snare loop 202 may provide stiffness to the distal end 214 of the snare loop 202, while the proximal end of the snare loop 202 may be stiffened by its interaction with shaft 102 and/or by its attachment with a distal end of the snare actuating element 302. In some embodiments, the proximal end of the snare loop 202 may be stiffened by increasing the thickness of the proximal end of the snare loop 202.

In one embodiment, the snare loop 202 may include sharpened outer edges, and may also comprise abrasive coatings or projections, such as barbs, saw teeth, or blades, although such protrusions should be sized to allow movement of the snare loop 202 through lumen 204.

Snare loop 202 may be configured for electrocauterization procedures. In such an embodiment, the snare loop 202 may be configured to carry sufficient electric current to generate the heat required for electrocautery. The snare loop 202 may be substantially hollow with holes disposed on a surface (e.g., an internal surface) to provide, e.g., irrigation or lubrication during electrocauterization or any other medical procedure. Furthermore, the snare loop 202 should be able to withstand repeated heat cycling without developing “hot spots” and breaking down. The general characteristics of suitable materials and configurations for snare loop 202 are known in the art. In one embodiment, the snare loop 202 may be made of a suitable biocompatible material, e.g., stainless steel or nitinol. On the other hand, the shaft 102 and the support truss 104 may be made from a variety of suitable biocompatible materials available in the art, such as nitinol, stainless steel, or polyimide. The chosen material may be based on desired stiffness, resilience, and other properties, as will be understood by those skilled in the art.

The shaft 102 and the support truss 104 can be made of the same or different types of materials, including those mentioned above, based on the degree of flexibility desired for each of those components for accessing the targeted tissue. The shaft 102 and the support truss 104 may be coated with a suitable friction reducing material such as TEFLON®, polyetheretherketone, polyimide, nylon, polyethylene, or other lubricious polymer coatings, to reduce surface friction with the surrounding tissues. The support truss 104 may be made of, or covered with, an insulating layer, for example, a hydrophilic layer of polymers known in the art, to prevent inadvertent cauterizing of surrounding tissue.

Further, the shaft 102, the support truss 104, and the snare loop 202 may be coated with an antimicrobial covering to inhibit any microbial growth on its surface. For instance, the coating may include an anti-bacterial covering, which may contain an inorganic antibiotic agent, disposed in a polymeric matrix that adheres the antibiotic agent onto the surface of the snare loop 202. Furthermore, a drug-releasing coating may be applied to the surface of the snare loop 202 for assisting in delivery of drugs to the targeted tissue during operation.

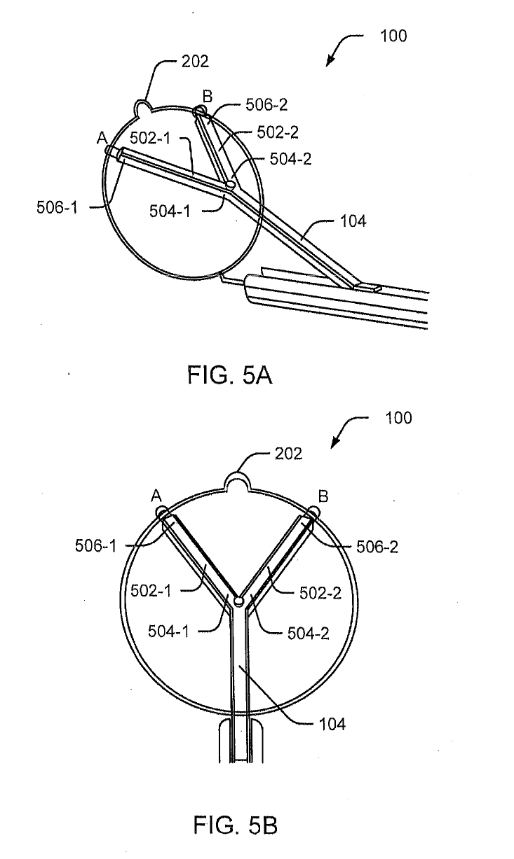

FIGS. 3A and 3B illustrate actuation of the exemplary retrieval device 100 from the collapsed configuration of FIG. 1 to the expanded configuration of FIGS. 2A-2C according to a first embodiment of the present disclosure. The retrieval device 100 may be used for severing and/or capturing tissue, such as, a polyp, or other target objects, for example, a kidney stone, from body lumens. In a collapsed configuration, the support truss 104 may be at least partially received within slot 208 of the shaft 102, while the snare loop 202 is located within the lumen 204 of the shaft 102, such that the distal end 214 of the snare loop 202 may be connected to the distal end 212 of the truss 104. The proximal end of the snare loop 202 may be connected to the snare actuating element 302, which operatively connects the snare loop 202 to a snare control device (not shown in the figures) such as an actuation handle, a control rod, or other conventional device. In certain embodiments, the truss 104 may be coupled to a truss actuating element 304 located within a lumen 306 inside the elongate shaft 102. The truss actuating element 304 may operatively connect the truss 104 to a truss control device 406 (FIG. 4A-4C), which drives the truss 104, discussed later. Further, the snare control device may be used for distally extending and proximally retracting the snare loop 202 with respect to the distal opening 206 of the shaft 102. The snare control device may be located in or received within the hub assembly of the endoscopic device 10.

In the illustrated embodiment, the snare actuating element 302 may be a solid or braided wire extending from snare loop 202 to the proximal end of shaft 102. The element 302 may be arranged in a way such that a proximally directed force on the element 302 closes snare loop 202 and may draw truss 104 toward the slot 208, and a distal force opens truss 104 and deploys snare loop 202. Thus, a distal force on the snare actuating element 302 may cause the support truss 104 to move radially away from the slot 208 while the truss 104 pivots about its the proximal end 210. The snare actuating element 302 may be made of a conductive material, such as nitinol, stainless steel, and polyimide, to serve as an electrical path for cautery.

One of skill in the art will understand that a stop element, for example, stop element 402 (FIG. 4A-4C), may be attached proximate to a proximal end of the snare loop 202 at a circumferential end of snare loop 202 opposite to the point of connection of the snare loop 202 with the truss 104. When the distal force is applied on the snare loop 202, the stop element 402 may be configured to provide limited distal movement of the snare loop 202 relative to the distal opening 206 of the shaft 102. For this task, the shaft 102 may be provided with protrusions (not shown) directed towards the lumen 204 of the shaft 102 for causing the stop element 402 to press against the protrusion to prevent further distal movement of a proximal portion the snare loop beyond a predetermined location, such as the distal opening 206 of the shaft 102.

A variety of mechanisms known in the art may be employed to selectively extend or retract the snare loop 202 relative to the distal opening 206. In one example, a simple push-pull mechanism may be employed using the snare actuating element 302 for either manually or automatically extending and/or retracting the snare loop 202 from or into the lumen 204 of the shaft 102. In the open configuration, as explained above, the snare loop 202 presents a target acquisition area based on the dimensions of the snare loop 202.

Referring to FIGS. 4A, 4B, and 4C, which illustrate operation of the exemplary retrieval device 400 according to a second embodiment of the present disclosure. In the second embodiment, a proximal portion of the truss 104 may be configured to advance distally within the slot 208 of the shaft 102 when the truss 104 is in the expanded configuration. Such distal movement of the truss 104 may be controlled with the help of a primary stopper 404 connected to a truss control device 406, which may be same or similar to the snare control device, such as an actuation handle. The primary stopper 404 may be employed to prevent the truss 104 from extending distally beyond a predetermined location within the slot 208 along the shaft 102. This predetermined location may be either proximate to or at the distal end of the shaft 102. In a first example, the primary stopper 404 may be contained within the slot 208 of the elongate shaft 102 such that the distal end 212 of the truss 104 is in communication with the primary stopper 404 in the collapsed configuration.

In a second example, the primary stopper 404 may be positioned at any location, for e.g., proximate to the proximal portion of the truss 104, along the length of the support truss 104 for restricting distal movement of the truss 104 beyond the predetermined location. In the above examples, a cross-section of the shaft 102, along with both the truss 104 and the primary stopper 404, may be configured to be substantially circular; however, other suitable cross-sectional shapes, for example, elliptical, oval, polygon, irregular, etc., may be employed.

In addition to the primary stopper 404, the retrieval device 400 may include a secondary stopper (not shown) configured to prevent the support truss 104 from retreating proximally within the slot 208, when the snare loop 202 is fully extended. One of skill in the art will understand that the primary stopper 404 and the secondary stopper may be manufactured of suitable size, shape, and material based on the amount of applied force and their location on the retrieval device 400 in communication with the support truss 104. It is contemplated that the slot 208 may be omitted in the embodiment of FIGS. 4A and 4B. In addition, the snare actuating element 302 may be operated using a snare control device for a controlled distal movement with the help of the stop element 402 in a manner similar to that discussed above.

During operation in the expanded configuration (discussed earlier), the truss 104 may be configured to be advanced distally by applying a distal force on either the snare actuating element 302, the truss actuating element 304, or both the snare actuating element 302 and the truss actuating element 304. In one example, a distal force applied on the snare actuating element 302 may distally extend the snare loop 202. The loop 202 may in turn pull the truss 104 distally due to the portion of the snare loop 202 being anchored to the distal end 212 of the truss 104. In another example, a simultaneous distal force may be applied on both the snare actuating element 302 and the truss actuating element 304 for causing the truss 104 to advance distally. It is also contemplated that the snare actuating element 302 may be held stationary, either by a user or by engagement of primary stopper 404, while truss actuating element 304 is actuated and vice versa.

Further, the snare actuating element 302 and the truss actuating element 304 may be applied with distal forces mutually exclusive to each other. For example, as illustrated in FIG. 3B, the support truss 104 may be stationary in the expanded configuration such that the snare loop 202 may extend distally and bow out away from the shaft 102 due to attachment of the snare loop 202 with the truss 104. A distal force applied on either the support truss 104 or the snare loop 202 may cause the angle between the snare loop 202 and the elongate shaft 102 to change.

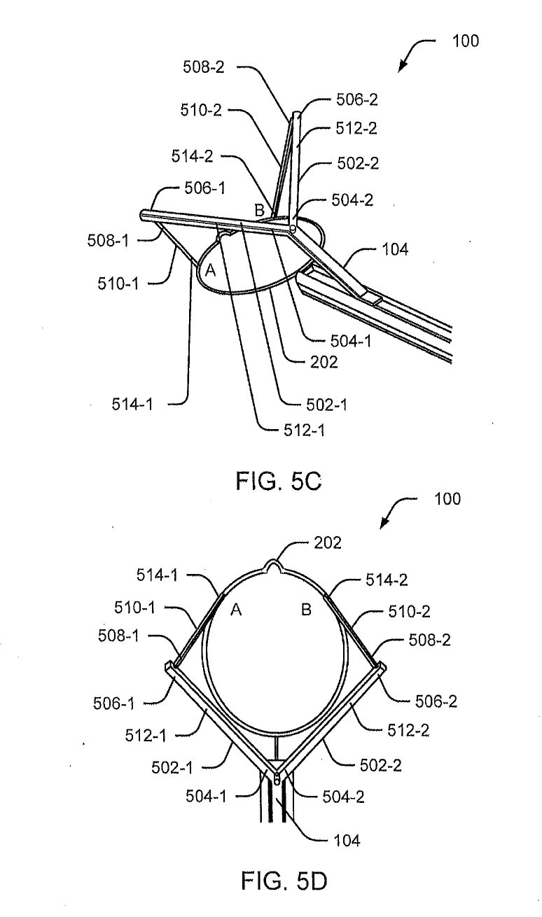

FIGS. 5A, 5B, 5C, and 5D illustrate the operation of the exemplary retrieval device 500 according to a further embodiment of the present disclosure. Here, a distal portion of the truss 104 may include a plurality of extension arms, such as extension arms 502-1 and 502-2, extending distally and at an angle to the longitudinal axis of truss 104. Each or at least one of the extension arms, for example, the extension arm 502-1, may be configured to pivot relative to the other extension arms, such as the extension arm 502-2. These extension arms 502-1, 502-2 and truss 104 may together form a Y-shaped member, with extension arms 502-1, 502-2 pivotally mounted at their proximal ends 504-1, 504-2. The pivoting force may be applied by the action of snare loop 202, as discussed below.

In one example, the proximal ends 504-1, 504-2 of the extension arms 502-1, 502-2 respectively may be pivotally attached to the distal portion of the truss 104 using one or more swivels or hinges. In another example, the extension arms 502-1, 502-2 may be formed integrally with truss 104, having proximal ends 504-1, 504-2 joined by a flexible strip of material enabling arms 502-1, 502-2 to pivot about their proximal ends 504-1, 504-2. The degree of flexibility of the extension arms 502-1, 502-2 may be predetermined based on a variety of factors, such as the ability of extension arms 502-1, 502-2 to fold about their proximal ends 504-1, 504-2 to meet each other longitudinally. This action of folding may facilitate to align arms 502 with the distal portion of the truss 104 so that the truss 104 can be arranged in a substantially linear configuration parallel to the snare loop 202, to enable it to retract into the slot 208 during the collapsed configuration. Alternatively, the truss 104 and extending arms 502-1 and 502-2 may extend from and retract into a second lumen (not shown) of the instrument shaft 102. The respective distal ends 506-1, 506-2 of the extension arms 502-1, 502-2 may be attached to the snare loop 202. For example, the distal end 506-1 may be attached to the snare loop 202 at point A and the distal end 506-2 may be attached to the snare loop 202 at point B.

FIGS. 5C and 5D illustrate another embodiment similar to the embodiment of FIGS. 5A and 5B. Extension arms 502 may further include a first rod 510-1 and a second rod 510-2. The distal end 514-1 of first rod 510-1 and the distal end 514-2 of second rod 510-2 may attach to point A and point B respectively along the circumference of the snare loop 202. The rods 510-1, 510-2 may be configured to pivot about their respective proximal ends 508-1, 508-2, which are located proximate to the distal ends 506-1, 506-2 of the extension arms 502-1, 502-2. The proximal ends 508-1, 508-2 of the rods 510-1, 510-2 may be attached to the distal ends 506-1, 506-2 of the extension arms 502-1, 502-2.

The embodiments of FIGS. 5A-D may operate similar to the embodiments discussed above. During operation to the expanded configuration, a distal force applied on a truss actuating element, such as truss actuating element 304, may provide a distal movement to extension arms 502-1, 502-2 (and therefore, to truss 104). Alternatively, a force applied to extend snare loop 202 from lumen 204 may provide a distal movement to the extension arms 502-1, 502-2 (or, more generally, to truss 104). The extension arms 502-1, 502-2 may be biased open to extend as snare loop 202 extends out from lumen 204. Alternatively, extension arms 502-1, 502-2 may be separately actuated to extend away from one another at an angle. Such distal movement of the extension arms 502-1, 502-2 may cause the snare loop 202 to extend and bow distally, while the extension arms 502-1, 502-2 move radially away from the elongate shaft 102. The snare loop 202 may bow in a plane different from that containing the elongate shaft 102.

In a second example, a distal force may be applied to the snare actuating element 302, causing the snare loop 202 to extend and bow distally in a plane lying at an angle to the axis of elongate shaft 102. This distal movement of snare loop 202 may responsively drive the distal end 506-1 of extension arm 502-1 and the distal end 506-2 of extension arm 502-2 to pivot outward as the extension arms 502-1, 502-2 move away from the elongate shaft 102 and the proximal portion of the truss 104 extends distally.

In a third example, as illustrated in FIG. 5C, a distal force applied on the truss 104 may cause the rods 510-1, 510-2 to move distally out of the elongate shaft 102. Such distal movement of the rods 510-1, 510-2 may responsively drive the snare loop 202 distally out of the elongate shaft 102 as well. During this expanded configuration, the pivoting proximal ends 508-1, 508-2 of the rods 510-1, 510-2 may apply a downward force on the snare loop 202 to improve snare traction. In some embodiments, the rods 510-1, 510-2 may drive the snare loop 202 into a plane parallel to the plane containing the elongate shaft 102. At this point, extension arm 502-1 may be oriented at an angle with the extension arm 502-2 to form, for example, a Y-shape along with the truss 104. Such orientation of the extension arms 502-1, 502-2 may ensure that the extension arms 502-1, 502-2 do not interfere with normal operation of the retrieval device 500. Further, the angle of approach of the snare loop 202 may be adjusted by maneuvering the length of extension arms 502-1, 502-2 and rods 508-1, 508-2 outside the elongate shaft 102.

In the expanded configuration, when the shaft 102 is manipulated to press the snare loop 202 against a target tissue, portions of the snare loop 202, which are unanchored to the truss 104 or the snare actuating element 302, may be deflected back into the longitudinal plane of the snare loop 202. Therefore, a force, which is capable of deflecting the snare loop 202, may be exerted against the tissue and distributed throughout the unanchored portions of the snare loop 202. It is to be noted that some force is applied directly from the portions of the snare loop 202 anchored to the truss 104 and those associated with the shaft 102. Such distributed normal forces applied against the tissue may increase the frictional force between the snare loop 202 and the tissue when the snare loop 202 is extended out from the distal opening 206. Further, these distributed normal forces may improve traction between the tissue and the snare loop 202 and decrease the likelihood of snare loop 202 slipping off of target tissue.

Accordingly, when a target tissue is to be captured, the snare loop 202 may be extended into the expanded configuration. The snare loop 202 is guided to receive the target tissue and is retracted into the lumen 204 of the shaft 102 until the target tissue is grasped. On the other hand, when the target tissue is to be severed, the snare loop 202 is actuated to receive the target tissue followed by full retraction of the snare loop 202 inside the lumen 204 of the shaft 102. Both operations may be performed with less downward force on the snare loop 202, improved traction of the snare loop 202 on the target tissue, and reduced risk of accidental damage to the surrounding tissues due to improved stiffness provided to the snare loop 202 by the support truss 104. Also, since the snare loop 202 extends at a predetermined angle from the shaft 102 based on dimensions of the snare loop 202, a better overhead view of the target tissue is possible.

It should be apparent that the retrieval devices 100, 400, 500 of the present disclosure are useful to perform surgical, diagnostic, and therapeutic procedures in a wide variety of bodily locations. For example, removal of polyps detected during a routine colonoscopy could quickly be accomplished using the method discussed above. Additionally, stones or unwanted deposits can be engaged and removed from a variety of body lumens such as ureters, bladders, or the urethra. These and other procedures can be accomplished within the scope of the present disclosure.

Embodiments of the present disclosure may be used in any medical or non-medical procedure, including any medical procedure where appropriate resection of undesired body tissue is required. In addition, at least certain aspects of the aforementioned embodiments may be combined with other aspects of the embodiments, or removed, without departing from the scope of the disclosure.

Other embodiments of the present disclosure will be apparent to those skilled in the art from consideration of the specification and practice of the embodiments disclosed herein. It is intended that the specification and examples be considered as exemplary only, with a true scope and spirit of the invention being indicated by the following claims.

What is claimed is:

1. A medical device for manipulating a tissue, the medical device comprising:
an elongate shaft having a proximal end, a distal end, and a first lumen extending therebetween, wherein the elongate shaft further includes a second lumen or an aperture disposed in an external surface of the elongate shaft;
an actuation member movably disposed within the first lumen, wherein the actuation member includes a distal end;
a support truss configured to be received within the first lumen, the second lumen, or the aperture; and
a snare loop having a first portion connected to a distal end of the support truss and a second portion connected to the distal end of the actuation member.

2. The medical device of claim 1, wherein a distal portion of the support truss includes a first extension arm attached to the support truss and a second extension arm attached to the support truss, and wherein the first extension arm forms an angle relative to the second extension arm.

3. The medical device of claim 2, wherein the first extension arm is configured to be pivotable relative to the second extension arm.

4. The medical device of claim 2, wherein a rod extends distally from each of the distal ends of the first extension arm and the second extension arm.

5. The medical device of claim 1, wherein the support truss is pivotally attached at a proximal end of the first lumen, second lumen, or aperture.

6. The medical device of claim 1, wherein the support truss is configured to move longitudinally within the first lumen, second lumen, or aperture.

7. The medical device of claim 1, wherein the support truss is driven by a truss actuating element located within the second lumen inside the elongate shaft.

8. The medical device of claim 1, wherein the support truss is a substantially rigid integral member.

9. The medical device of claim 1, wherein a proximal end of the support truss is operably connected to the shaft adjacent a proximal portion of the first lumen, second lumen, or aperture.

10. A medical device for manipulating a tissue, the medical device comprising:
an elongate member defining an elongate opening at a distal end leading to a distally facing opening;
a substantially rigid member disposed within the elongate opening; and
an expandable wire loop configured to transition between a collapsed configuration and an expanded configuration, wherein a portion of the expandable wire loop is connected to the substantially rigid member.

11. The medical device of claim 10, wherein the rigid member is configured to extend distally within the elongate opening.

12. The medical device of claim 10, wherein the rigid member is pivotally attached at the proximal end of the elongate opening.

13. The medical device of claim 10, wherein a distal portion of the rigid member includes a first extension arm attached to the rigid member and a second extension arm attached to the rigid member, and wherein the first extension arm forms an angle relative to a second extension arm.

14. The medical device of claim 13, wherein the first extension arm is configured to be pivotable relative to the second extension arm.

15. The medical device of claim 10, wherein the rigid member is driven by an actuating element located within a first lumen of the elongate member and the wire loop is driven by a wire loop actuating element located within a second lumen of the elongate member.

16. The medical device of claim 10, further comprising a stopper configured to restrict distal movement of the rigid member beyond a predetermined location within the elongate opening.

17. The medical device of claim 10, further comprising a stopper element configured to restrict distal movement of the expandable wire loop beyond a predetermined location within the elongate member during the expanded configuration.

18. The medical device of claim 10, wherein the medical device includes an elongate introduction sheath including imaging and illumination.

19. The medical device of claim 10, wherein the distal end of the elongate shaft includes an opening in communication with the first lumen and the elongate opening.

20. A method for manipulating tissue, the method comprising:
introducing a medical device into the body, the medical device comprising an elongate shaft having a proximal end, a distal end, and a first lumen extending therebetween, wherein the distal end of the elongate shaft includes an opening in communication with the lumen, and wherein the elongate shaft further includes a second lumen or aperture disposed in an external surface of the elongate shaft;
advancing an actuation member of the device to advance the medical device to a desired location within the body, the actuation member movably disposed within the first lumen, wherein the actuation member includes a distal end;
advancing a snare loop of the device to receive tissue, the snare loop having a first portion connected to a distal end of a support truss and a second portion connected to the distal end of the actuation member; and
supporting the snare loop with the truss support while advancing the snare loop, the truss support configured to be received within the first lumen, the second lumen, or the aperture, wherein a proximal end of the support truss is operably connected to the shaft adjacent a proximal portion of the first lumen, second lumen, or aperture.
